(19) United States
(12) Patent Application Publication
(10) Pub. No.: US 2024/0342415 A1
(43) Pub. Date: Oct. 17, 2024

(54) FACE MASKS AND BAG-VALVE MASK RESUSCITATOR SYSTEMS FOR VENTILATION OF PATIENTS

(71) Applicant: Spirare Medical LLC, Providence, RI (US)

(72) Inventors: PAUL AKERMAN, Providence, RI (US); Chetan Dusane, Mountain View, CA (US); Courtney Kowalsky, Oyster Bay, NY (US); Ya-Sing ` Lin, Irvine, CA (US); Harry Yu, New York, NY (US)

(21) Appl. No.: 18/683,221

(22) PCT Filed: Aug. 10, 2022
(86) PCT No.: PCT/US2022/039940

§ 371 Date: Feb. 12, 2024

Related U.S. Application Data
(60) Provisional application No. 63/231,480, filed on Aug. 10, 2021.

(51) Int. Cl. A61M 16/06; A61M 16/00; A61M 16/08

(52) U.S. Cl. CPC A61M 16/0616; A61M 16/0084; A61M 16/0816

(57) ABSTRACT

A face mask that can be used for the ventilation of patients such as in bag-valve-mask resuscitator systems is provided. The face mask can be used to form a tight seal with the patient's mouth and nose, while also creating an open airway. The face mask includes a jaw (chin)-lifting member that can be used to position the patient's head in a tilted position. The invention also encompasses bag-valve mask resuscitator systems and methods for using bag-valve mask resuscitators in the ventilation of patients. In one embodiment, the bag-valve mask resuscitator is placed so that the bag is positioned over the midline of the face of the patient.

### CROSS-REFERENCE TO RELATED APPLICATIONS

This application claims the benefit of U.S. Provisional Patent Application No. 63/231,480 filed Aug. 10, 2021, the entire disclosure of which is hereby incorporated by reference.

### BACKGROUND OF THE INVENTION

### Field of the Invention

The present invention relates generally to a face mask that can be used for ventilation of patients such as in bag-valve mask resuscitator systems. The invention also encompasses bag-valve mask resuscitator systems and methods for using such systems in ventilating patients.

### Brief Review of the Related Art

In emergency, critical-care, surgical, and other cases, medical personnel commonly use bag-valve mask resuscitators to deliver oxygen or anesthetic gasses under positive pressure to a non-breathing patient. In recent years, the number of cases where bag-valve mask resuscitators are needed to treat patients have significantly increased. These cases generally fall in the categories of cardiac arrest or respiratory arrest. The bag-valve mask resuscitators normally include a resilient, compressible bag having an air intake valve at one end and a breathing/exhaust valve at the other end. The bag is generally shaped like a football. The bag is squeezed to create pressure and cause air to flow through the breathing/exhaust valve. A face mask covers the nose and mouth of the patient. The face mask is coupled to the breathing/exhaust valve and used to administer air to the non-breathing patient.

In general, these face masks include a lower cuff (rim) and a dome extending upwardly from the cuff. The cuff makes direct contact with the face of the patient during ventilation. The medical provider (for example, emergency paramedics, nurses, technicians, physicians, and the like) grips the mask and places it over the patient's face so there is a tight seal between the mask and nose/mouth. When positioning the mask on the patient, the lower and wider end of the mask is placed over the mouth, while the narrower end (bridge) is placed over the patient's nose.

There are generally two methods that the medical provider uses to administer the air and ventilate the patient: a) a two-person method, wherein one provider holds the face mask with two hands and applies downward pressure to create a tight seal; while another medical provider squeezes the bag; and b) a one-person method, wherein a single medical provider holds the face mask with one hand using a grip commonly known as a C-E grip to create a seal; and the same provider uses the other hand to squeeze the bag. For example, a medical provider can hold the face mask with his/her left hand while squeezing the bag with their right hand to develop positive pressure air flow to the patient.

It is important that a tight seal be formed between the face mask and the patient's mouth and nose. If a tight seal is not created, air will tend to leak out of the mask. At the same time, an open airway must be created and maintained. Normally, the patient is positioned so that he/she is lying on their back. By placing and keeping the patient's head in a tilted position, an open airway for the patient can be created. The jaw (chin) is thrust upwardly to establish good air flow so the patient can be fully ventilated. If the one-person technique is used, the single provider must use one hand to apply sufficient downward pressure to create a seal between the mask and patient's face; while also keeping the patient's head in a tilted position and lifting the chin up and forward. Simultaneously, the provider must use the other hand to squeeze the bag and ventilate the patient.

In contrast, when the two-hand technique is used, the operator uses both hands to create pressure on opposite sides of the mask and achieve a tight seal with the face. Pressure exerted on the mask with both hands can more easily be controlled. This is an advantageous feature of the two-hand technique. However, when the medical provider needs to use two hands to apply pressure on the mask, this means that a second medical provider must be available to compress the bag. This is disadvantageous because it means that another provider is needed to work the bag-valve mask and this takes the provider away from performing other medical procedures.

In recent years, bag-valve masks that can be used with one hand have been developed as described in the patent literature. For example, Kileny et al., U.S. Pat. No. 9,415,181 discloses a face mask for bag-valve-mask resuscitators having a contoured outer surface accommodating left and right-handed use of the mask. According to Kileny '181, the operator can use the mask in an ambidextrous or two-handed manner, and the mask is easy for use for clinicians with smaller hands. Kileny '181 suggests that the clinician will have less muscle fatigue using this face mask to maintain a seal against the patient's face than is required using other face masks. The contoured dome allows the clinician's hand to be efficient and not waste muscle energy on movement or finger positions.

Saad, U.S. Pat. No. 8,393,324 discloses a face mask for bag-valve-mask resuscitators that the operator can use with his/her hand and arm in an anatomically favorable position. The mask in Saad '324 includes extensions that form vertical and horizontal surfaces. These extensions help the operator compress and orient the face mask so that it can be sealed with the patient's face.

Matioc, U.S. Pat. No. 6,651,661 discloses a face mask for bag-valve-mask resuscitators that is ergonomic and comfortable for the operator to use. The mask in Saad '324 includes a depressed channel that surrounds the outer periphery of the rim. The channel includes a right groove, posterior groove, and left groove that defines an asymmetrical and continuous concave structure for the operator to grasp with their hand.

One drawback with using conventional bag-valve masks is that the medical provider can have difficulty forming a tight seal between the face mask and the patient's mouth and nose, while also creating and keeping an open airway. If a tight seal with the patient's face is not created, air will tend to leak out of the mask. If the patient's head is not placed in a tilted position with the jaw (chin) pointed upwardly, there tends to be poor air flow and ventilation. In some cases, it can take considerable strength and coordination to perform these steps. Performing this procedure can lead to the medical provider suffering muscle fatigue and possible injury. The procedure can be particularly difficult for medical providers having a small hand size. Some of the more common reasons for muscle fatigue and/or the leaking of air out of the face mask include: a) over exertion of the fingers, hand, and forearm flexor muscles due to the provider using the C-E grip; b) poor balancing of the face mask over the patient's face because the bag is positioned off-center and the provider must use one hand must to squeeze the bag, while using the other hand to press downwardly on the mask.

In view of the drawbacks with some conventional bag-valve masks, there is a need for a face mask that medical providers can more easily use to properly adjust the patient's head while maintaining a tight seal between the mask and patient's face. The present invention provides such a face mask. In addition, there may be some problems with handling conventional bag-valve mask resuscitator systems. As discussed further below, in a traditional one-person method, the bag is not positioned over the facial midline of the patient. This makes it difficult for the operator to balance the bag which rests in one hand and the face mask which rests in their other hand. The present invention provides improved bag-valve mask resuscitator systems. The face masks and bag-valve mask resuscitator systems and methods of the present invention provide additional features, benefits, and advantages as described further below.

### SUMMARY OF THE INVENTION

The present invention relates generally to a face mask for ventilating patients, comprising: a) a dome member, wherein the dome member has a deformable lower rim for forming a seal between the dome and face of a patient such that the dome forms an enclosed cavity, and an upper air connector port for delivering air to the enclosed cavity; and b) a lower jaw-lifting member for lifting the jaw of the patient when pressure is applied to the jaw-lifting member by a user. In one embodiment, the jaw-lifting member extends from the neck of the air connector port. In another embodiment, the jaw-lifting member extends from the lower rim of the face mask. In yet another embodiment, the jaw-lifting member extends from the dome of the face mask. The jaw-lifting member can be fixed or moveable, for example, the jaw-lifting member can be attached by a hinge so that it pivots. The air connector port can be connected to any suitable ventilation system such as a bag-valve mask resuscitator or ventilator tube.

In one preferred embodiment, the air connector port is tilted at an angle. Preferably, the air connector port is tilted at an angle is in the range of about 30° to about 60°. In another embodiment, the air connector port preferably has a length in the range of about 2 to about 6 inches. In yet another embodiment, the face mask has a relatively low contoured profile. For example, the height of the face mask from the lower edge of the rim to the upper edge of the dome can be in the range of about 1.5 to about 2.75 inches.

The invention further includes a bag-valve mask resuscitator comprising; a) a compressible bag that can be manually squeezed to deliver air to a patient; b) a first end having an inlet valve through which air is delivered to the bag; c) a second opposing end; and d) a breathing connector that is centered between the first and second ends through which air is delivered to the patient; wherein the breathing connector has a coupling for connection to a face mask that is placed on the patient and an exhaust outlet so that air is allowed to flow from the bag and into the patient and exhaled air from the patient is directed into the exhaust outlet. In another embodiment, there are two inlet valves located on one end of the bag through which air is delivered to the bag. The air is forced through the breathing connector and into the patient.

The invention also includes a method for using a bag-valve mask resuscitator on a patient. In one example, the method comprises the steps of: a) providing a bag-valve mask resuscitator, the resuscitator comprising a dome member, the dome member having a deformable lower rim for forming a seal between the dome and face of a patient such that the dome forms an enclosed cavity and an upper air connector port for delivering air to the enclosed cavity; b) having an operator position the bag-valve mask resuscitator so that the bag is positioned over the midline of the face of the patient; c) having the operator use one hand to apply downward pressure to create a seal between the mask and patient's face; d) having the operator place the patient's head in a tilted position to create an open airway for the patient; and e) having the operator use the other hand simultaneously to squeeze the bag and ventilate the patient.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description in connection with the accompanying drawings in which:

FIG. 1 is a schematic drawing showing a bag-valve mask resuscitator of the prior art;

FIG. 2 is a perspective view of one embodiment of the bag-valve mask resuscitator of the present invention including a chin-lifting member and a centered breathing connector coupled to the bag;

FIG. 2A is a schematic drawing showing a medical provider administering air to a patient using the bag-valve mask resuscitator shown in FIG. 2, wherein the bag is positioned over the midline of the face of the patient;

FIG. 3 is a schematic drawing showing a medical provider gripping a bag-valve resuscitator of the prior art using a C-E grip;

FIG. 4 is a schematic drawing showing a medical provider gripping one embodiment of a bag-valve resuscitator of the present invention using a centralized grip;

FIG. 5 is a side view of another embodiment of the bag-valve mask resuscitator of the present invention showing the mask disposed over the face of a patient showing a hinged chin lifting member;

FIG. 6 is a side view of another embodiment of the bag-valve mask resuscitator of the present invention;

FIG. 7 is a side view of a bag-valve mask resuscitator of the prior art;

FIG. 8 is a side view of another embodiment of the bag-valve mask resuscitator of the present invention showing an angled air connector port;

FIG. 9 is a perspective view of another embodiment of the bag-valve mask resuscitator of the present invention disposed over the face of a patient showing the low profile contour of the mask;

FIG. 10 is a perspective view of a bag-valve mask resuscitator of the prior art disposed over the face of a patient;

FIG. 11 is a perspective view of one embodiment of the bag-valve mask resuscitator system of the present invention showing the bag positioned over the midline of the face of the patient;

FIG. 12 is a perspective view of another embodiment of the bag-valve mask resuscitator system of the present invention showing a centered breathing connector and central air outflow as the bag is being positioned over the midline of the face of the patient; and

FIG. 13 is a perspective view another embodiment of the bag-valve mask resuscitator system of the present invention showing a centered breathing connector and central air outflow.

### DETAILED DESCRIPTION OF THE INVENTION

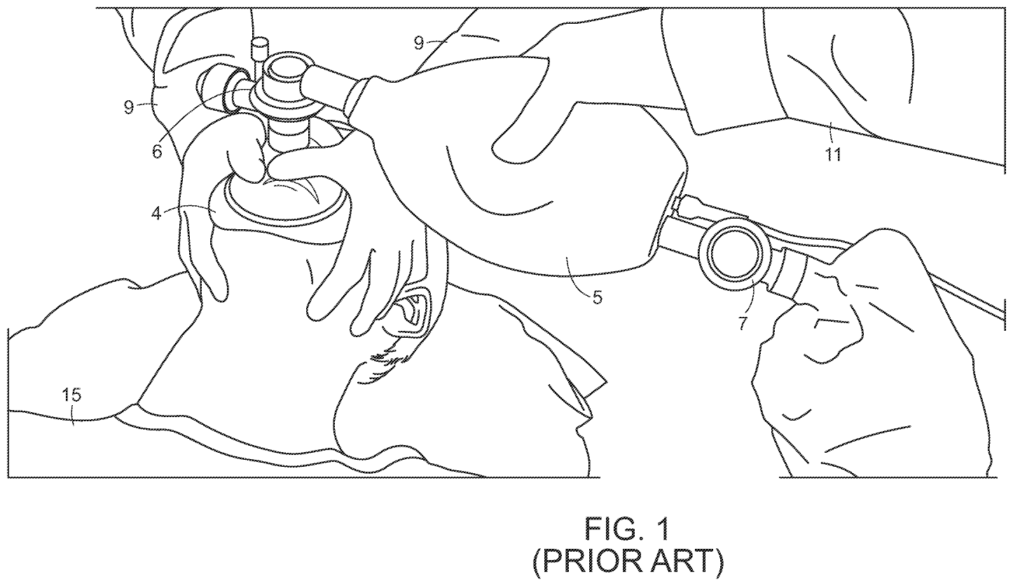

Referring to the Figures, where like reference numerals are used to designate like elements, a conventional bag-valve mask resuscitator is generally shown in FIG. 1. As discussed above, bag-valve mask resuscitators are commonly used to deliver oxygen or anesthetic gasses under positive pressure to a non-breathing patient. As shown in FIG. 1, a traditional two-person method can be used to administer oxygen to the patient. In this method of the prior art, one medical provider (9) holds the face mask (4) with two hands and applies downward pressure to create a seal over the nose and mouth of the patient (13); while another medical provider (11) squeezes the compressible bag (5).

The first medical provider (9) uses both hands to create pressure on opposite sides of the mask (4) and achieve a tight seal. Because the first medical provider (9) needs to use two hands to apply pressure on the mask, the second medical provider (11) must be available to compress the bag (5). This means that the second provider (11) is unable to attend to other medical procedures. As shown in FIG. 1, the bag-valve mask resuscitator normally includes a resilient, compressible bag (5) having a breathing/exhaust valve (6) at one end and an air intake valve (7) at the other end. The bag (5) is squeezed to create pressure and cause air to flow through the valves to ventilate the patient (15).

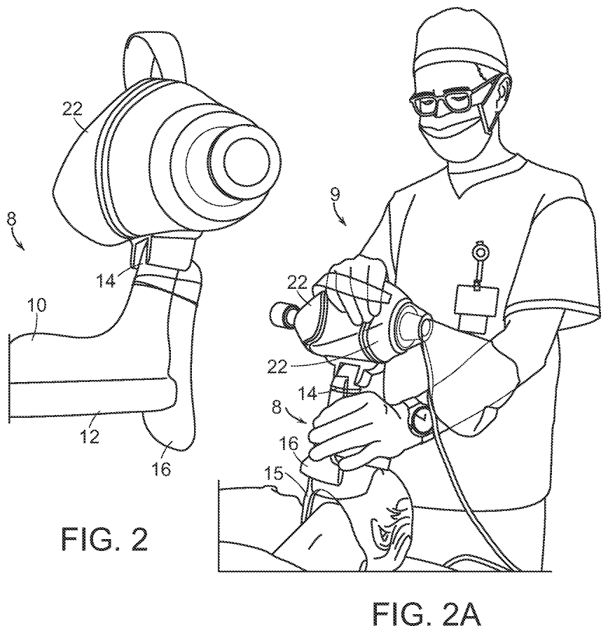

Referring to FIGS. 2 and 2A, one embodiment of the face mask of the present invention, which is generally indicated at (8), is shown. The face mask (8) includes an upper dome member (10). The dome member (10) has a deformable lower rim (12) for forming a seal between the dome and face of a patient. The lower rim (12) makes direct contact with the face of the patient (15) during ventilation. The upper dome (10) forms an enclosed air cavity (not shown). The dome (10) can be made of any suitable material such as a soft plastic and is normally transparent. The lower rim (12) also can be made of a soft plastic material. Other deformable materials such as a soft foam can be used to form the lower rim (12) and in other instances, the rim can be inflated. The lower rim (12) forms a temporary mold over the patient's face, particularly the nose and mouth. The dome member (10) further includes an upper air connector port (14) for delivering air to the enclosed cavity. In practice, the medical provider (9) grips and seals the mask (8) to the patient's face as discussed further below.

As shown in FIG. 2A, it is important that a tight seal be formed between the face mask (8) and the patient's mouth and nose. If a tight seal is not created, air will tend to leak out of the mask (8). This tight seal is created by pressing downwardly on the face mask (8) with uniform pressure. At the same time, an open airway for the patient must be created and maintained. This is done by keeping the patient's head in a constantly tilted position as he/she is in a resting position lying on their back.

As discussed in further detail below, one advantage of the face mask (8) of the present invention is that the mask preferably includes a jaw (chin)-lifting member (16). With the face mask (8), the jaw (chin) is thrust upwardly using the jaw (chin)-lifting member (16) and the patient's airway can be kept open.

### Gripping of Face Mask

As discussed above and illustrated in FIG. 1, in a traditional two-person method, one medical provider (90 holds the face mask (4) with two hands and applies downward pressure to create a tight seal; while another medical provider (11) squeezes the bag (5).

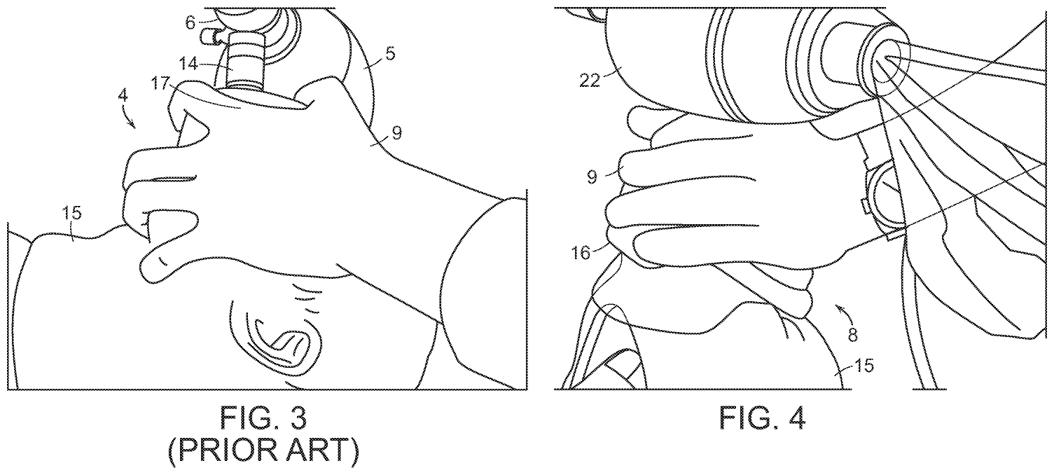

In other cases, a one-person method is used. Referring to FIG. 3 in a traditional one-person method, a single medical provider holds the face mask (4) with one hand using a grip commonly known as a C-E grip (17) to create a seal; and the same provider uses the other hand to squeeze the bag (5). It can be difficult for a single operator to grip the mask with one hand and squeeze the bag with the other hand using this conventional technique. Operating both the face mask and the bag can lead to muscle fatigue and an inefficient ventilation process.

Referring to FIG. 4, in one embodiment of the face mask (8) of the present invention, a single operator can hold the mask on the patient's face using a distinctive grip with one hand, while squeezing the bag (22) with the other hand. More particularly, as shown in FIG. 4, the operator can use a specialized centralized grip, wherein he/she wraps their hand completely around the relatively long air connector port (not shown) and partially around the dome (10) of the mask. This gripping technique is further shown in FIG. 10. More particularly, the operator grasps the air connector port (14) with their hand so that the index, middle, ring, and small fingers press against a front side of the port, and the thumb presses against the back side of the port. The single operator can use one hand to apply sufficient downward pressure to create a seal between the mask (8) and patient's face; while also keeping the patient's head in a tilted position and lifting the chin up and forward.

One advantageous feature of the face mask (8) of the present invention is that the mask preferably includes a jaw (chin)-lifting member (16) as discussed further below, and this piece helps lift the patient's chin up. Simultaneously, the operator can use the other hand to squeeze the bag and ventilate the patient. In other words, for example, a medical provider can hold the face mask in place by wrapping his/her left hand completely around the relatively long air connector port (not shown) while squeezing the bag (22) with their right hand to deliver air to the patient.

This new gripping method, wherein the provider grasps the air connector port (14) and around the dome (10) applies downward pressure on the mask (8) has several advantages. For example, the provider can use a single hand to create equal levels of pressure on opposite sides of the mask. This equal pressure helps create a tight seal between the mask and face. Pressure exerted on the mask with both hands can more easily be controlled. Secondly, the major arm muscles provide the necessary downward forces on the face mask. Thus, there is generally greater forces applied to the mask and a tight seal can be more easily achieved. Furthermore, there is less strain placed on the muscles using this centralized gripping method versus the traditional C-E grip. Also, the clinician's hands can be more efficient and there is less strain exerted on the outstretched fingers using the centralized gripping method of the present invention.

As discussed above, the projecting air connector port (14) can be of any suitable length (height) and diameter, but it preferably has a long length. More particularly, the air connector port (14) preferably has a size of about 2 to about 6 inches and more preferably about 3 to about 5 inches. The relatively long air connector port (14) provides the user with more leverage when using the face mask (8) on the patient. The provider can apply a centralized-grip around the air connector port (14) and dome (10) with one hand while simultaneously using the jaw-lifting member (16) to lift-up and hold the jaw as discussed in further detail below. The operator has more leverage to press downwardly on the mask (8) in this procedure using the mask of the present invention.

More particularly, as shown in FIG. 4 the medical provider can achieve a tight seal between the face mask (8) and face of the patient by applying pressure in two areas. In FIG. 4, the provider is shown using a bag-valve resuscitator (22) to ventilate the patient. The medical provider applies upper pressure on the dome (10) and lower pressure to the jaw-lifting member (16). As the provider uses one hand to hold the mask (8) and wraps his/her hand around the air connector port (14), pressure is applied to the dome (10). Pressure is applied downwardly on the dome (10) and the mask (8) is sealed to the face. At the same time, the provider applies lower pressure to the jaw-lift (chin-lift) (16) as discussed further below.

### Jaw (Chin)-Lifting Member

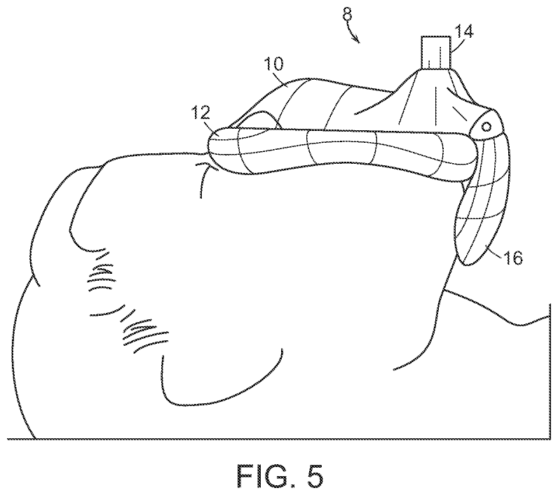

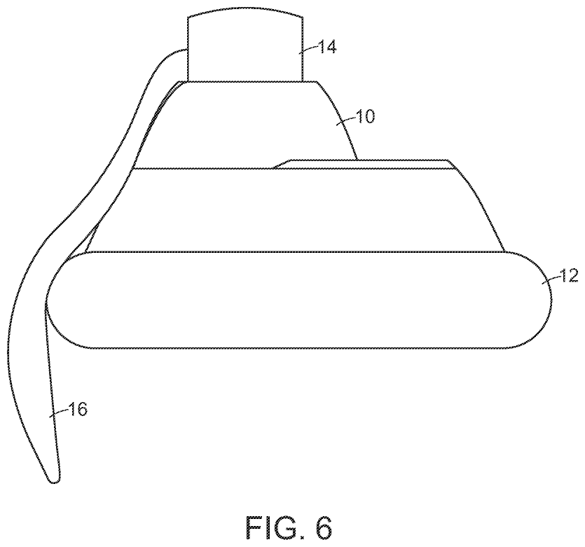

As discussed above, the face mask (8) of the present invention preferably includes a lower jaw (chin)-lifting member (16). This helps the provider lift the jaw of the patient when the operator applies pressure to the jaw-lifting member. In this way, air is delivered to the patient for breathing. Referring to FIG. 5, one embodiment of a face mask (8) having the lower jaw (chin)-lifting member (16) of the present invention is shown. In FIG. 6, another embodiment of a face mask (8) of the present invention is shown. As shown in FIGS. 5 and 6, the upper region of the dome (10) has a relatively large surface area so that a user can more easily grasp the face mask (8) and apply pressure for sealing the mask to the patient's face. This feature is particularly beneficial for users having a small hand size. Also, the dome (10) is relatively soft and has a low-profile contour making it easier to handle than many conventional face masks.

By pressing upwardly on the jaw-lifting member (16) and lifting the jaw, the medical provider can keep the patient's head in a tilted position. The jaw-lifting member (16) supports the jaw. The medical provider uses the jaw-lifting member (16) to hold and lift the chin. With a single hand and in one continuous motion, the medical provider is able to press down on the face mask and lift up and hold the chin in a tilted position. The jaw-lifting member (16) of this invention provides a unique mechanical advantage. With the jaw-lifting member (16), there is better leverage for lifting the chin. The medical provider can press down on the face mask (8) to form a tight seal with the patient's face while lifting and holding the chin in a tilted position using the jaw-lifting member (16). Both steps can be accomplished in just a single motion. This technique helps provides for a tight facial seal and good air flow so the patient can be fully ventilated.

Using the jaw-lifting member (16) of the face mask (8) of this invention helps create a tight seal between the mask and face of the patient. Moreover, the jaw-lifting member (16) helps open the airway of the non-breathing patient. It is critical that the jaw be forced upwardly in order for the airway to be opened. With the face mask (8) of the present invention, the medical provider can use one hand to create a seal and lift the jaw of the patient to open their airway. At the same time, the medical provider can use their other hand to squeeze the bag-valve (22) and administer air to the patient. The present invention provides a face mask (8) that medical providers can easily use to adjust the patient's head to the proper position while maintaining a tight seal between the mask and patient's face.

As the provider uses one hand to hold the mask (8) and wraps his/her hand around the air connector port (14), pressure is applied to the dome (10). Pressure is applied downwardly on the dome (10) and the mask (8) is sealed to the face. At the same time, the provider applies lower pressure to the jaw-lift (chin-lift) (16) with their middle, ring, and small fingers. As the medical provider applies this pressure, the jaw of the patient is pushed upwardly and held in a tilted position. The jaw is lifted and the mouth is pressed tightly against the face mask (8). As the jaw is held in this propped-up position, the airway is opened clearly so the patient can be fully ventilated.

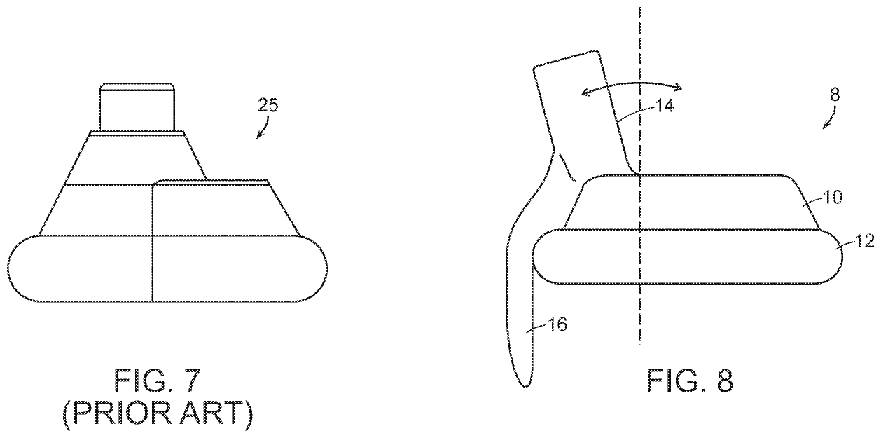

In FIG. 7, a conventional face mask (25) is shown. The face mask (25) does not contain a jaw-lifting member (16) of this invention. One problem with such traditional bag-valve masks (25) is that the medical provider can have difficulty forming a tight seal between the face mask and the patient's mouth and nose, while also creating and keeping an open airway. The patient's head must be place in tilted position with the jaw (chin) pointed upwardly so there can be good air flow. The provider must use their fingers, hand, and forearm muscles to tilt the patient's head and this can lead to muscle fatigue. Turning to FIG. 8, using the jaw-lifting member (16) of the face mask (8) of this invention helps make it easier to tilt the head and force the jaw upwardly and open the airway of the non-breathing patient (25).

In contrast to the traditional face mask (25) shown in FIG. 7, the operator can easily use one hand on the face mask (8) of the present invention to create a seal and lift the jaw of the patient. At the same time, the operator can use their other hand to squeeze the bag-valve (22) and administer air to the patient. As shown in FIG. 8, the present invention provides a face mask (8) that medical providers can easily use to adjust the patient's head to the proper position while maintaining a tight seal between the mask and patient's face.

### Angled Air Connector Port (Inlet)

More particularly, referring to FIG. 8, in this embodiment of the present invention, the air connector port (14) is angled relative to the longitudinal axis of the face mask. That is, the air connector port (14) is tilted at an angle. Preferably, the angle is in the range of about 20° to about 70° and more preferably, the angle is in the range of about 30° to about 60°. This angled air connector port (14) provides several advantages. For example, this angled air connector (14) allows the operator to better reach his/her fingers around the chin of the patient and force the chin upwardly. Secondly, this face mask (8) with the angled air connector gives the operator more hand-resting area on the dome (10). Thirdly, the face mask (8) with the angled air connector means that greater nose ridge-sealing force can be applied to the patient's face.

It should be recognized that the jaw-lifting member (16) can have any suitable dimensions and shape. For example, the jaw-lifting member (16) can have a concave-shaped lower portion similar in shape to a cup so that the chin can rest and be supported by this cup like area. It also is recognized that the jaw-lifting member (16) can be joined to and extend from other portions of the face mask (8), for example, the jaw-lifting member (16) can extend from the lower rim (12) of the face mask in other embodiments. In yet another embodiment, the jaw-lifting member extends from the dome of the face mask. The jaw-lifting member can be fixed or moveable, for example, the jaw-lifting member can be attached by a hinge so that it pivots.

The face mask (8) may be molded or otherwise manufactured as one piece with the integrated jaw-lifting member (16). In other embodiments, the jaw-lifting member (16) may be designed as a separate piece that is securely fastened to the face mask (8). That is, the face mask (8) may be retro-fitted with the jaw-lifting member (16). Adhesives, screws, rivets, hinges, connectors, clips, snap buttons, or any other suitable fastening means may be used to fasten the jaw-lifting member (16) to the face mask (8).

### Contour of Face Mask

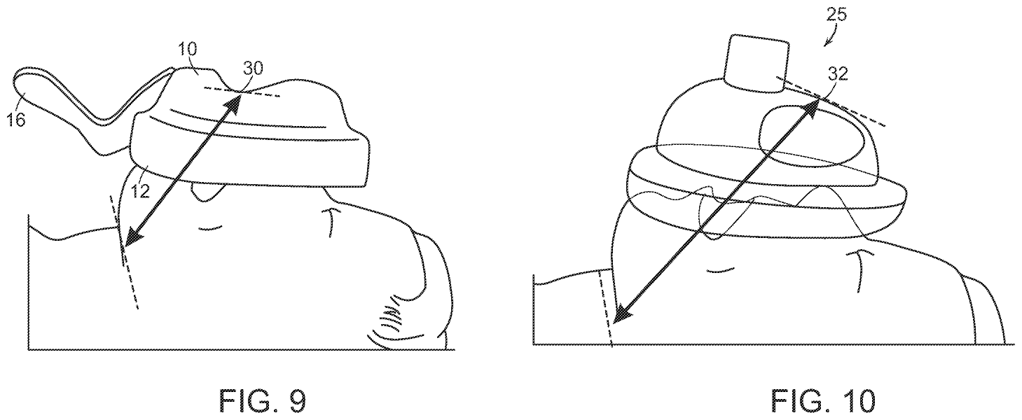

Another advantageous feature of the face mask of the present invention is that the nose bridge is lowered. In FIG. 9, one embodiment of a face mask (8) of the present invention is shown. The face mask has a relative low profile. The nose/mouth bridge (30) that covers the nose and mouth is relatively close to the patient's face. In contrast, as shown in FIG. 10, a conventional face mask (25) is shown. Traditional face masks has a relatively high profile. Here, the nose/mouth bridge (32) that covers the nose and mouth is relatively far away from the patient's face. One problem with such conventional bag-valve masks is that the medical provider can have difficulty forming a tight seal between the face mask and the patient's mouth and nose, while also creating an open airway.

### Centering of Bag Over Facial Midline of Patient

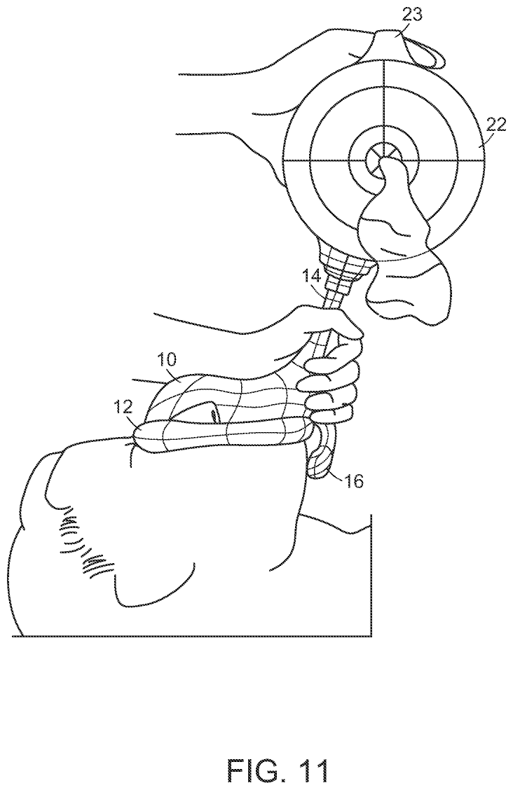

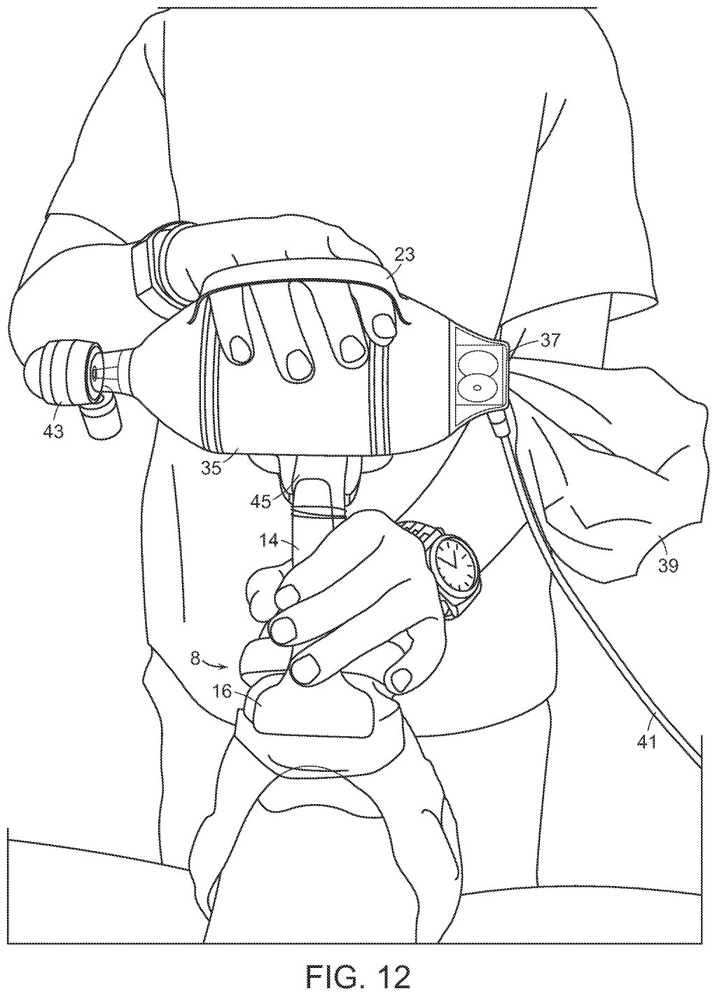

Turning to FIGS. 11-13, in another embodiment, the invention further includes a bag-valve mask resuscitator system (assembly) comprising; a) a compressible bag (35) that can be manually squeezed to deliver air to a patient; b) a first end having an inlet valve (37) through which air is delivered to the bag (35); c) a second opposing end; and d) a breathing connector (45) that is centered between the first and second ends of the bag (45) through which air is delivered to the patient. This assembly provides a centralized air outflow to the patient. The breathing connector (45) has a coupling for connection to a face mask (8) that is placed on the patient and an exhaust outlet (47). In this way, air is allowed to flow from the bag (35) and into the patient and exhaled air from the patient is directed into the exhaust outlet (47). The bag (35) is made of a generally resilient, compressible material and is shaped like a football. The bag (35) can be referred to as an “insufflator bag.” The bag (35) is compressed manually to create pressure and cause air to flow through the centralized breathing connector (45). When the operator's hand-pressure on the bag (35) is released, the bag returns to its original shape.

As discussed above, in this embodiment, the compressible bag (35) includes an inlet valve (37) on its first end through which air is delivered into the bag. The bag (35) also includes a second end that is opposite the first end. In conventional bag-valve mask resuscitators, the breathing connector is located on the second end of the bag. However, in the present invention, the breathing connector (45) is positioned at the center of the compressible bag (35). Thus, there is a centered breathing connector (45) and a central air outflow to the patient. As discussed further below, this central positioning of the breathing connector (45) provides several benefits. The breathing connector (45) is connected to the compressible bag (35). When the bag (35) is manually compressed, air flows into the coupling and attached face mask (8) that is sealed over the patient's nose and mouth. The inlet valve (37) allows air to enter the bag (35) when it is in a resting, steady state; however, it prevents air from escaping out of the bag through the inlet when the bag is being compressed. When the patient exhales, the exhaled air flows from the breathing connector (45) through the exhaust port (47).

In yet another embodiment, the there are two inlet valves located at one end of the bag through which air is delivered to the bag. The air is forced through the breathing connector, which is located at the center point and thickest area of the bag, and into the patient.

The present invention also encompasses a method for using a bag-valve mask resuscitator system (assembly) on a patient. The method comprises the steps of: a) providing a bag-valve mask resuscitator; b) positioning the bag-valve mask resuscitator so that the bag is positioned over the midline of the face of the patient; c) using one hand to apply downward pressure to create a seal between the mask and patient's face; and d) using the other hand simultaneously to squeeze the bag and ventilate the patient. The facial midline of the patient is an imaginary line that bisects the face at the center thereof. The centering of the bag over the midline of the face of the patient is an important step as discussed further below.

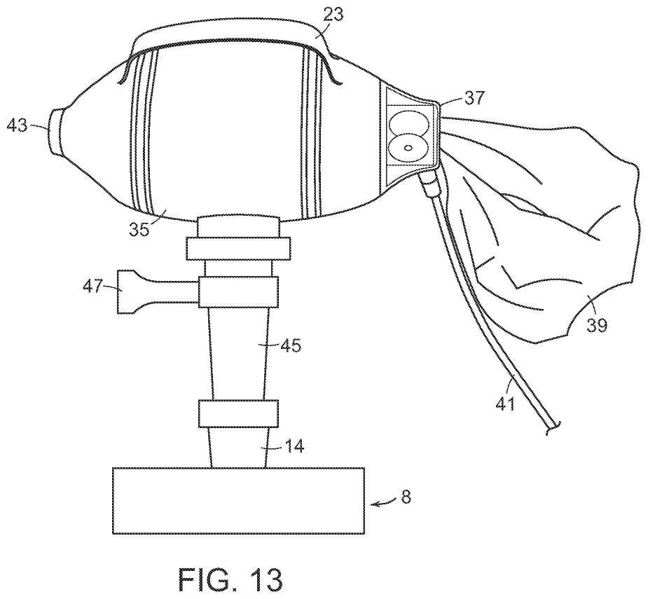

Referring to FIGS. 12 and 13, different embodiments of a bag-valve mask resuscitator system (assembly) of the present invention are shown in more detail. As discussed above, the compressible bag (35) includes an inlet valve (37) on its first end through which air is delivered into the bag. There is an oxygen reservoir bag (39) attached to the inlet valve (37); and an oxygen line (41) feeds the reservoir bag (39). On the opposing second end of the compressible bag (35), there is an end cap or pressure relief valve (43). The breathing connector (45) is positioned at the center of the compressible bag (35). Thus, there is a centered breathing connector (45) and a central air outflow that forces air into the patient when the bag is being squeezed.

In one preferred method, the face mask used in the centered bag-valve mask resuscitator system comprises a dome member, the dome member having a deformable lower rim for forming a seal between the dome and face of a patient such that the dome forms an enclosed cavity and an upper air connector port for delivering air to the enclosed cavity. In another preferred method, the face mask used in the centered bag-valve mask resuscitator system further comprises a lower jaw-lifting member for lifting the jaw of the patient when pressure is applied to the jaw-lifting member by a user so that air can be delivered to the patient.

Turning back to FIGS. 1 and 3, in a traditional one-person method, the bag (5) is not positioned over the facial midline of the patient. Rather, the bag (5) is positioned off to the side of the patient. In some cases, these traditional bags (5) can be connected by a long tubing to the mask. This makes it difficult for the operator to balance the bag (5) which rests in one hand and the face mask (4) which rests in their other hand. In contrast, as shown in FIGS. 11-13, it has been found that having a centered bag-valve mask resuscitator system (assembly) with a central air outflow and positioning the bag (35) over the facial midline of the patient provides several advantages.

For example, some advantages with centering the bag (35) over the facial midline of the patient means that no off-center forces are applied to the bag (35). Rather, the operator can simply apply the necessary forces directly downward on the bag (35) and face mask (8). In this method, the operator's hand that compresses the bag (35) rests directly over the midline of the patient's face. Furthermore, the operator does not have to balance the bag (35) on the left or right side of the patient's face; while simultaneously balancing the mask (8) on the center of the patient's face. The operator does not need to worry about counter-balancing the system and applying sufficient pressure to the bag and face mask. With the centered bag-valve mask resuscitator system of this invention, the operator can more easily apply equal pressure to the points of the bag (35) and face mask (5), thus ventilating the patient and sealing the mask.

In recent years, there has been an increasing number of cardiac and respiratory arrests, wherein the patient becomes non-breathing. In such cases, the patient needs air ventilation immediately. Conventional bag-valve mask resuscitators used to ventilate patients have some drawbacks. Such resuscitators can be difficult to handle. The medical provider needs to form a tight seal between the face mask and the patient's mouth and nose, while also creating and keeping an open airway. The bag needs to be squeezed to ventilate the patient. Medical providers may need to have relatively large sized hands to perform these steps adequately. Performing these steps can lead to the provider developing muscle fatigue and injury. In turn, this can lead to inefficient ventilation of the patient and loss of life.

Also, as discussed above, in a traditional one-person method, the bag is not positioned over the facial midline of the patient. This makes it difficult for the operator to exert a sufficient amount of force as he/she tries to balance the bag which rests in one hand and the face mask which rests in their other hand. Thus, in a conventional two-person method, one provider typically holds the face mask with two hands and applies downward pressure to create a tight seal; while another medical provider squeezes the bag.

The face mask, bag-valve mask resuscitator systems, and methods of the present invention overcome the above-discussed drawbacks. The face masks and bag-valve mask resuscitator systems and methods of this invention can be effectively used for ventilating patients.

It should be understood the terms, “first”, “second”, “top”, “bottom”, “upper”, “lower”, “upper”, “downward”, “right”, “left”, “anterior”, “posterior”, and the like are arbitrary terms used to refer to one position of an element based on one perspective and should not be construed as limiting the scope of the invention.

It also should be understood that the face masks, bag-valve mask resuscitator systems, methods, materials, and constructions described and illustrated herein represent only some embodiments of the invention. It is appreciated by those skilled in the art that various changes and additions can be made to the device and constructions without departing from the spirit and scope of this invention. It is intended that all such embodiments be covered by the appended claims.

We claim:

1. A face mask, comprising:
a dome member, the dome member having a deformable lower rim for forming a seal between the dome and face of a patient such that the dome forms an enclosed cavity and an upper air connector port for delivering air to the enclosed cavity; and
a lower jaw-lifting member for lifting the jaw of the patient when pressure is applied to the jaw-lifting member by a user so that air can be delivered to the patient.

2. The face mask of claim 1, wherein the jaw-lifting member extends from the neck of the air connector port.

3. The face mask of claim 1, wherein the jaw-lifting member extends from the lower rim of the face mask.

4. The face mask of claim 1, wherein the air connector port has a length in the range of about 2 to about 6 inches.

5. The face mask of claim 1, wherein the air connector port is coupled to a bag-valve.

6. The face mask of claim 1, wherein the air connector port is coupled to a ventilator tube.

7. A face mask, comprising:
a dome member, the dome member having a deformable lower rim for forming a seal between the dome and face of a patient such that the dome forms an enclosed cavity and an upper air connector port for delivering air to the enclosed cavity, wherein the air connector port is tilted at an angle; and
a lower jaw-lifting member for lifting the jaw of the patient when pressure is applied to the jaw-lifting member by a user so that air can be delivered to the patient.

8. The face mask of claim 7, wherein the air connector port is tilted at an angle is in the range of about 30 to about 60 degrees.

9. The face mask of claim 7, wherein the air connector port has a length in the range of about 2 to about 6 inches.

10. The face mask of claim 7, wherein the height of the face mask from the lower edge of the rim to the upper edge of the dome is in the range of about 1.5 to about 2.75 inches.

11. The bag-valve mask resuscitator comprising;
a) a compressible bag that can be manually squeezed to deliver air to a patient;
b) a first end having an inlet valve through which air is delivered to the bag;
c) a second opposing end; and
d) a breathing connector that is centered between the first and second ends of the bag through which air is delivered to the patient; wherein the breathing connector has a coupling for connection to a face mask that is placed on the patient and an exhaust outlet so that air is allowed to flow from the bag and into the patient and exhaled air from the patient is directed into the exhaust outlet.

12. The bag-valve mask resuscitator of claim 11, wherein the face mask comprises a dome member, the dome member having a deformable lower rim for forming a seal between the dome and face of a patient such that the dome forms an enclosed cavity and an upper air connector port for delivering air to the enclosed cavity.

13. The bag-valve mask resuscitator of claim 12, wherein the face mask further comprises a lower jaw-lifting member for lifting the jaw of the patient.

14. The bag-valve mask resuscitator of claim 12, wherein the dome member is made of a plastic material.

15. The bag-valve mask resuscitator of claim 12, wherein the bag has an upper surface and lower surface, the breathing connector being fastened to the lower surface and a handle being fastened to the upper surface.

16. A method for using a bag-valve mask resuscitator on a patient, comprising the steps of:
a) providing a bag-valve mask resuscitator;
b) positioning the bag-valve mask resuscitator so that the bag is positioned over the midline of the face of the patient;
c) having an operator used one hand to apply downward pressure to create a seal between the mask and patient's face; and
d) having the operator use the other hand simultaneously to squeeze the bag and ventilate the patient.

17. The method of claim 16, wherein the face mask comprises a dome member, the dome member having a deformable lower rim for forming a seal between the dome and face of a patient such that the dome forms an enclosed cavity and an upper air connector port for delivering air to the enclosed cavity.

18. The bag-valve mask resuscitator of claim 16, wherein the face mask further comprises a lower jaw-lifting member for lifting the jaw of the patient.

19. The method of claim 17, wherein the air connector port is tilted at an angle is in the range of about 30 to about 60 degrees.

20. The method of claim 17, wherein the air connector port has a length in the range of about 2 to about 6 inches.
